(19) United States
(12) Patent Application Publication
(10) Pub. No.: US 2017/0137804 A1
(43) Pub. Date: May 18, 2017

(54) Method of Stem Cell Preparation and Administration

(71) Applicant: Rakesh N. Chettier, West Jordan, UT (US)

(72) Inventor: Rakesh N. Chettier, West Jordan, UT (US)

(73) Assignees: Rakesh N. Chettier, West Jordan, UT (US); Michael R. Schramm, Perry, UT (US)

(21) Appl. No.: 15/421,186

(22) Filed: Jan. 31, 2017

Related U.S. Application Data
(60) Provisional application No. 62/295,042, filed on Feb. 13, 2016.

(51) Int. Cl. C12N 15/10; A61K 35/28; A61K 9/00; C12Q 1/68

(52) U.S. Cl. CPC C12N 15/102; C12Q 1/6883; A61K 35/28; A61K 9/0085; C12N 2510/00; C12Q 2600/156

(57) ABSTRACT

The present invention is a method of testing and or treating SCs in preparation for administration of such SCs and a method of administering the prepared SCs to a patient. The method has particular application in avoiding the administration of SCs having or being predisposed to a predetermined genetic condition. The method may be used in allogeneic administration of the prepared SCs but the method has particular usefulness in the autogeneic administer of the prepared SCs.

### CROSS REFERENCE TO RELATED APPLICATIONS

This nonprovisional utility patent application claims the benefit under 35 U.S.C. §119(e) of U.S. provisional application No. 62/295,042 filed Feb. 13, 2016 which is incorporated herein in its entirety by this reference.

### FIELD OF THE INVENTION

The present invention relates to methods for use in preparing stem cell compositions and for use in administering such compositions to patients.

### BACKGROUND OF THE INVENTION

Stem cells (SC) or compositions containing SCs (including mesenchymal stem cells or MSC and Multipotent Stromal Cells) are known in the art (see Appx A) as are various methods of using such SCs, to treat a variety conditions including genetic and non-genetic conditions in humans (see Appx C). Methods of editing genes and in particular, a gene editing method entitled the CRISPR Cas System (CRSP) is also known in the art (see Appx B). Further, various genetic conditions or diseases are known in the art as are methods of testing for predisposition to such conditions and associated treatments for such conditions. Exemplary methods are disclosed for instance in U.S. Pat. No. 8,641,738 for Method of Treating Scoliosis Using a Biological Implant issued to Ogilvie at al., US publication 2015/0337373 for Genetic Markers Associated with Degenerative Disc Disease and Uses Thereof published to Chettier at al., and U.S. Pat. No. 8,932,993 for Method of Testing for Endometriosis and Treatment Therefor issued to Ward at al., all of which are incorporated herein in their entirety by this reference.

### SUMMARY OF THE INVENTION

The present invention is a method of testing and or treating SCs in preparation for administration of such SCs and a method of administering the prepared SCs to a patient. The method has particular application in avoiding the administration of SCs having or being predisposed to a predetermined genetic condition. The method may be used in allogeneic administration of the prepared SCs but the method has particular usefulness in the autogeneic administer of the prepared SCs.

### DETAILED DESCRIPTION OF THE INVENTION

Reference throughout this specification to “one embodiment” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

A first embodiment of the invention is a method of preparing an SC composition and administering the SC composition to a patient. The method preferably includes the following steps. A quantity of SCs is obtained from a donor. The SCs are genetically screen (e.g. genetically sequenced and analyzed). At least one marker associated with the existence or predisposition to at least one genetic condition is identified. The genetic sequence of the SCs is altered such that the SCs no longer include a marker associated with the existence or predisposition to at least one genetic condition. The SCs are administered to a patient.

A second embodiment of the invention is a method of preparing an SC composition and administering the SC composition to a patient. The method preferably includes the following steps. A patient is determined to be genetically predisposed to degenerative disk disease (DDD) or to have clinical indications of DDD or both. A quantity of SCs is obtained from the patient. The SCs are genetically screen (e.g. genetically sequenced and analyzed). At least one marker associated with the existence or predisposition to DDD (AKA an increased risk or so-called “causative” marker) is identified. The genetic sequence of the SCs is edited via CRSP such that the SCs no longer include a marker associated with the existence or predisposition to DDD and even more preferably such that the SCs come to include a marker associated with the absence or non-predisposition to DDD (AKA a decreased risk or so-called “protective” marker). The SCs are caused to initiate differentiation into the tissue of choice, namely disk tissue. The SCs are administered to the patient via injection into at least one disk of the patient. It is noted that because the SC administration is an autogeneic administration, the SCs have a very high likelihood of acceptance (i.e. will not be rejected by the patient's immune system) and yet the SCs will stimulate regeneration of disk material that is not predisposed to DDD.

It is noted that a process substantially similar to the above may be used in administering SCs to address virtually any genetic condition. For instance, autogeneic administration of SCs that differentiate to non-endometriosis (cured) tissue may be administered to the endometrial area of a patient, and in particular to a site where a lesion has been surgically treated via laparoscopy. Likewise, autogeneic administration of SCs that differentiate to non-scoliotic (cured) tissue may be administered to the spinal area of a patient who is otherwise genetically predisposed to scoliosis curve progression.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A treatment method comprising obtaining at least one SC from a patient determined to have or be predisposed to at least one genetic condition, identifying at least one marker associated with an increased risk of said condition in said at least one SC, editing the genetic sequence of said at least one SC via CRSP such that at least one of the following is performed comprising elimination of said condition increased risk associated marker from said at least one SC and introduction of a condition decreased risk associated marker to said at least one SC, causing said at least one SC to initiate differentiation into a predetermined tissue, administering said edited at least one SC to said patient.

2. The method of claim 1 wherein said at least one genetic condition defines endometriosis.

3. The method of claim 1, wherein if said at least one genetic condition defines DDD, said predetermined tissue defines spinal disk tissue and said administering defines injecting said edited at least one SC into at least one spinal disk of said patient, and wherein if said at least one genetic condition defines scoliosis curve progression said administering defines injecting said edited at least one SC into the spine of said patient, and if said at least one genetic condition defines endometriosis said administering defines administering said edited at least one SC into the abdominal/pelvic region of said patient.

4. A treatment method comprising identifying at least one marker associated with an increased risk of a genetic condition in an SC editing the genetic sequence of said at least one SC such that at least one of the following is performed comprising elimination of said genetic condition increased risk associated marker from said at least one SC and introduction of a genetic condition decreased risk associated marker to said at least one SC, causing said SC to initiate differentiation into a predetermined tissue, administering said edited SC to a patient.

5. The method of claim 4, wherein said editing is performed via CRSP.

6. The method of claim 4, wherein said administering defines at least one of autogeneic administering and allogeneic administering.

7. The method of claim 4, wherein differentiation of said edited SC into a predetermined tissue is initiated prior to administering said edited SC to said patient.

8. The method of claim 4, wherein said genetic condition defines endometriosis.

9. The method of claim 4, wherein if said condition defines DDD, said predetermined tissue defines spinal disk tissue and said administering defines injecting said edited SC into at least one spinal disk of said patient, and wherein if said condition defines scoliosis curve progression said administering defines injecting said edited SC into the spine of said patient, and if said condition defines endometriosis said administering defines administering said edited SC into the abdominal/pelvic region of said patient.

10. A treatment method comprising editing the genetic sequence of an SC such that at least one of the following is performed comprising elimination of a genetic condition increased risk associated marker from said SC and introduction of a genetic condition decreased risk associated marker to said SC, and administering said edited SC to a patient.

11. The method of claim 10, wherein said editing is performed via CRSP.

12. The method of claim 10, wherein said administering defines at least one of autogeneic administering and allogeneic administering.

13. The method of claim 10, wherein differentiation of said edited SC into a predetermined tissue is initiated prior to administering said edited SC to said patient.

14. The method of claim 10, wherein said genetic condition defines endometriosis.

15. The method of claim 10, wherein if said condition defines DDD, said predetermined tissue defines spinal disk tissue and said administering defines injecting said edited SC into at least one spinal disk of said patient, and wherein if said condition defines scoliosis curve progression said administering defines injecting said edited SC into the spine of said patient, and if said condition defines endometriosis said administering defines administering said edited SC into the abdominal/pelvic region of said patient.

16. A treatment method comprising editing the genetic sequence of an SC such that at least one of the following is performed comprising elimination of a genetic condition increased risk associated marker from said SC and introduction of a genetic condition decreased risk associated marker to said SC.

17. The method of claim 16, wherein said SC is administered to a patient.

18. The method of claim 17, wherein said administering defines at least one of autogeneic administering and allogeneic administering.

19. The method of claim 17, wherein differentiation of said edited SC into a predetermined tissue is initiated prior to administering said edited SC to said patient.

20. The method of claim 16, wherein said editing is performed via CRSP.

21. The method of claim 16, wherein said genetic condition defines endometriosis.

22. The method of claim 16, wherein if said condition defines DDD said edited SC is administered into at least one spinal disk of a patient, and wherein if said condition defines scoliosis curve progression said edited SC is administered into the spine of a patient, and if said condition defines endometriosis said edited SC is administered into the abdominal/pelvic region of a patient.
